(19) United States
(12) Patent Application Publication
(10) Pub. No.: US 2004/0203004 A1
(43) Pub. Date: Oct. 14, 2004

(54) Diagnostic apparatus and method

(76) Inventors: Hans Ulrich Bernard, Irvine, CA (US); Fuk Loi Li, Singapore (SG); Vinay Badal, Singapore (SG)

Correspondence Address: CLARK & ELBING LLP, 101 FEDERAL STREET, BOSTON, MA, 02110, US

(21) Appl. No.: 10/410,969

(22) Filed: Apr. 10, 2003

(51) Int. Cl. C12Q 1/68

(52) U.S. Cl. 435/6

(57) ABSTRACT

A method of diagnosing cervical cancer in a patient infected with human papillomavirus-16 (HPV-16) is disclosed. The method comprises determining the presence or absence of methylation on CpG sites in the HPV-16 genome in a sample obtained from the patient. The more CpG sites on which methylation is absent the more indicative it is of cervical cancer. Also disclosed are primers and kits useful for carrying out the method. It is preferred that the methylation state of the CpG site at position 7862 of the HPV-16 genome is determined.

### FIELD OF THE INVENTION

The present invention relates to the diagnosis of cervical cancer, and also to methods of assessing the progression of cervical cancer. Furthermore, the present invention relates to DNA primers and kits for use in diagnosing cervical cancer.

### BACKGROUND OF THE INVENTION

Human papillomavirus-16 (HPV-16) and related HPV types are carcinogenic, and persistent HPV-16 infection is the primary cause of cervical cancer. Most women become infected by HPVs, and while some of these infections progress malignantly, most remain subclinical or lead only to precursor lesions (zur Hausen, H. and Schiffman, M. H. et al.)

The factors that determine these outcomes are poorly understood. Transformation by HPVs depends on the HPV oncoproteins E6 and E7, whose transcription is modulated by numerous transcription factors and epigenetic mechanisms (Bernard, H. U.). Tumor progression may result from stimulated oncoprotein expression through transcriptional induction by steroids (Chan, W. K., et al.) by deletion of transcriptional silencers (May, M. et al.) and by integration of HPV genomes into cellular DNA (Baker, C. C. et al., and Stünkel, W., et al.).

At present, early detection of cervical cancer is an important component of its treatment. Typically this occurs by programmes for screening so-called “Pap” smears (i.e. Papanicolaou-stain cervical smears). The problem with this type of cervical cancer screening is that skilled personnel are required in order to examine smears. Furthermore, the results can be unreliable because the visual identification of transformed cells is inherently a subjective determination, which is open to opinion. Accordingly, there is a need for alternative methods of screening for cervical cancer, in particular methods which are more objective and reliable.

One alternative method of diagnosing cervical cancer is disclosed in U.S. Pat. No. 6,355,424. The expression levels of particular genes of the HPV genome are monitored and compared in order to assess the progression of HPV infection from benign tumor to malignant growth. Similarly, PCT publication no. WO01/42792 discloses a method of assessing whether a patient is afflicted with cervical cancer by comparing the level of expression of particular sequence markers with the normal level of expression of the markers, a significant difference between the two levels being indicative of the presence of cervical cancer. The sequence markers comprise sequences present in the HPV genome. Despite the existence of these alternative methods of screening for cervical cancer, there is still a need for further screening methods.

An area of study that has been implicated in tumorigenesis is DNA methylation. Mammalian cells are able to modify their genome DNA sequences by covalently adding a methyl group to cytosine residues. This can occur at the five position of the cytosine ring in locations where the cytosine is directly 5′ of a guanine residue (i.e. at a CpG dinucleotide). In mammals, DNA-methylation is mediated by a family of DNA-[cytosine-5] methyltransferases (DNMT1, DNMT3a and DNMT3b) (Okano, M., et al. and Rhee, I. et al.) and occurs at 2-4% of all cytosines located 5′ to guanine (CpG) (Bird, A. P.). CpG dinucleotides are present only at 20% of the statistically expected frequency in mammalian genomes (Schorderet, D. F. et al.) suggesting adverse functional consequences of methylation as a cause for their loss during evolution. However, in certain regions, known as “CpG islands”, the presence of these dinucleotides is at the statistically expected level.

CpG methylation leading to gene silencing is involved in X-chromosome inactivation and genomic imprinting (Paulsen, M. et al.). The rare case of tissue specific gene expression mediated by CpG methylation was recently confirmed (Futscher, B. W. et al.). Furthermore, CpG methylation is required for stabilization of the genome against recombination, implicated by preferential methylation of repeated sequences (Yoder, J. A., et al). During carcinogenesis, global hypomethylation and regional (i.e. CpG island) hypermethylation (Herman, J. G. et al) are frequently observed. CpG methylation mediates its functions in gene expression by either preventing transcription factors from recognizing their cognate binding sites (Robertson, K. D. et al. and Thain, A., et al.) or by proteins that bind methylated DNA (MeCPs) (Hendrich, B et al.) which recruit HDAC1 (histone deacetylase) to condense adjacent chromatin.

A study into the extent of viral DNA methylation in non-virus producing benign and malignant tumors induced by the shope (rabbit) papilloma virus found that the methylation patterns correlated with the degree of neoplasia (Wettstein, F. O. et al). However, the study found that, in general, the degree of methylation was higher in carcinomas than in papillomas (i.e. benign tumors). Similarly, another study into shope papilloma virus (SPV) DNA in benign and malignant rabbit tumors found that 10-40% of the CpG sites were methylated in papillomas, 30-80% in primary carcinomas and more than 90% in transplantable carcinomas (Sugawara, K. et al). Thus these studies indicate that the presence of methylation in SPV DNA is indicative of carcinomas.

PCT Publication No. WO02/059347 discloses a method of diagnosing a cell proliferative disorder of breast tissue by determining the methylation status of nucleic acids obtained from a subject. However, this publication relates only to findings in regard to genes involved in breast cancer and does not indicate how, or whether it is possible, to diagnose other types of cancer on the basis of methylation status.

As regards the control of HPV16, cis-responsive elements that regulate E6 and E7 oncogene transcription are spread throughout the long control region (LCR), an 850 bp segment between the L1 and E6 genes. Transcription starts at the E6 promoter P97, which is regulated by one binding site for Sp1 and two for the HPV encoded factor E2 (Tan, S. H., et al., and Demeret, C. et al.). The activity of P97 is stimulated by an enhancer with binding sites for several cellular factors including AP1, NF1, and the progesterone receptor (Tan et al., Chou et al., Apt, D. et al. and Gloss, B. et al.). Two specifically positioned nucleosomes can form over the enhancer and promoter (Stünkel, W. et al.) and repress transcription, when they are modified by histone deacetylases (HDACs). HDACs are associated with CDP, which binds a silencer between the enhancer and promoter (O'Connor, M. J. et al.). When HPV genomes integrate into cellular DNA during progression to malignancy, a nuclear matrix attachment region (MAR) located downstream of P97 becomes a strong transcriptional stimulator (Stünkel et al.). A study has been conducted into the methylation status of HPV-1a DNA and found a non-random of methylation (Burnett et. al.). It was speculated that DNA methylation might therefore be involved in the control of HPV-1a gene expression but the study was inconclusive.

It is thought that integration of HPV into the host genome is an early and important event in HPV linked cervical carcinogenesis. It has also been hypothesised that host-specific DNA methylation may play a functional role in HPV gene regulation (Rosl et al). It has also been suggested that viral transcription can be suppressed by methylation of HPV-16 regulatory region by suppressing the binding of a cellular transcription factor (List H. J. et al.). It has also been speculated that the degree to which integrated HPV DNA is CpG methylated might correlate with the tumorigenic potential of the host cells (Thain et al.). A study has been conducted into the methylation status of HPV-1a DNA and found a non-random pattern of methylation (Burnett et al.). It was speculated that DNA methylation might therefore be involved in the control of HPV-1a gene expression but the study was inconclusive.

Furthermore, methylation of HPV genomes in situ has never been studied, although there is evidence for its potential involvement in regulating HPV transcription. Firstly, CpGs are underrepresented in HPVs similar to their human host. Secondly, when in vitro methylated HPV-16 DNA was transfected into cells, it was not expressed (Rosl et al.) in a similar way to the EcoRI-resistant HPV-16 DNA studied in one specific cell line (List et al.).

The present inventors have now completed a study of CpG methylation of HPV-16 in cell lines and in clinical samples. They have found that HPV-16 DNA is an efficient target for DNA methylation, and that preferentially methylated regions are asymmetrically distributed over the genome. They have also found that methylation occurs at CpGs overlapping with the enhancer and promoter of the HPV-16 genome, and that the frequency of CpG methylation decreases during progressive stages of cancer, indicating that CpG methylation is involved in the etiology of cervical cancer and is a useful diagnostic marker.

It is therefore an object of the invention to provide an alternative or improved method of diagnosing cervical cancer or assessing the progression of cervical cancer. It is also an object of the invention to provide an alternative or improved kit for diagnosing cervical cancer or assessing the progression of cervical cancer.

### SUMMARY OF THE INVENTION

According to a first aspect of the present invention, there is provided a method of diagnosing cervical cancer in a patient infected with human papillomavirus comprising the steps of:

1) providing a sample containing the human papillomavirus genomic DNA from the cervical cells of the patient; and

2) determining the presence or absence of methylation on CpG sites in the human papillomavirus genome in the sample, wherein the more CpG sites on which methylation is absent the more indicative it is of cervical cancer.

Conveniently, the human papillomavirus is HPV-16.

Preferably, the HPV-16 genome has eleven CpG dinucleotides between positions 7498 and 161 and step 2 comprises determining the presence or absence of methylation at said eleven CpG dinucleotides.

Advantageously, the presence of methylation at all of said eleven CpG dinucleotides is indicative of the absence of cervical cancer.

Conveniently, there are CpG dinucleotides at positions 37, 43, 52 and 58 in the HPV-16 genome, the absence of methylation in at least one of said CpG dinucleotides being indicative of the presence of at least a precursor lesion at stage CIN I.

Preferably, the HPV-16 genome has a CpG dinucleotide at position 7862 and wherein the absence of methylation at position 7862 is indicative of at least a precursor lesion of level CIN I.

Advantageously, step 2) comprises the step of digesting the human papillomavirus genome with a methylation sensitive endonuclease.

Conveniently, step 2) further comprises the step of:

adding primers to the sample and effecting the PCR process, the primers being at either end of an amplicon containing at least one CpG site susceptible to cleavage by the methylation sensitive endonuclease and;

analysing the results of the PCR process to determine whether said at least one CpG site was cleaved.

Preferably, the primers are SEQ. ID NOS. 19 and 20.

Advantageously, the methylation sensitive endonuclease is McrBC endonuclease

Conveniently, step 2) comprises selectively modifying the cytosine residue of said eleven CpG dinucleotides depending upon the methylation status of the CpG.

Preferably, step 2) further comprises, after selectively modifying the cytosine residue, adding primers to the sample and effecting the PCR process, the primers being at either end of an amplicon containing the at least one CpG site.

Advantageously, the primers are SEQ ID NOS. 27 and 28.

Preferably, step 2) further comprises the step of sequencing at least a portion of the human papillomavirus genome containing the at least one CpG site after the step of selectively modifying the cytosine residue of the said eleven CpG dinucleotides, preferably using the primers from the PCR process.

Advantageously, the step of selectively modifying the cytosine residue comprises adding sodium bisulfite to the sample in order to modify unmethylated cytosine residues to uracil residues.

Conveniently, step 2) further comprises the steps of adding at least one detection probe capable of hybridising to said at least one CpG site with a first melting temperature and capable of hybridising to the site with a second, different melting temperatures when the cytosine residue of the CpG site has been modified; and monitoring the hybridisation of the detection probe to the portion of the human papillomavirus genome containing the CpG site at a range of temperatures after the modifying step.

Preferably, step 2) further comprises the steps of: adding to the sample at least one anchor probe capable of hybridising to a position on the human papillomavirus genome adjacent the at least one detection probe, the detection probe comprising one of a donor dye capable of absorbing light at a first wavelength and an acceptor dye capable of emitting light at a second wavelength, the anchor probe comprising the other of the donor dye and the acceptor dye, the donor and acceptor dyes being such that they are capable of fluorescence resonance energy transfer when they are in close proximity to one another; exposing the sample to light at the first wavelength and detecting the emission of light at the second wavelength over the range of temperatures.

Advantageously, the human papillomavirus is HPV-16, further comprising the steps of dividing the sample into eleven sub-samples; adding to each sub-sample a detection probe and a corresponding anchor probe, the detection probes being capable of hybridising to a CpG site at positions 7535, 7554, 7677, 7683, 7695, 7862, 31, 37, 43, 52 and 58, respectively, on the HPV-16 genome; exposing each sub-sample to light at the first wavelength and detecting light emitted at the second wavelength from each sub-sample over the range of temperatures.

According to a second aspect of the present invention, there is provided a method of assessing the progression of cervical cancer in a patient infected with HPV-16, comprising the steps of:

1) providing a sample containing the HPV-16 genomic DNA from the cervical cells of the patient; and

2) detecting the methylation state of at least the CpG dinucleotide at position 7862 of the HPV-16 genome, the lack of methylation at said dinucleotide indicating the presence of at least a precursor lesion of cervical cancer.

Conveniently, the HPV-16 genome has eleven CpG dinucleotides between positions 7498 and 161 and step 2) further comprises the steps of detecting the methylation state of the eleven CpG dinucleotides of the HPV-16 genome between positions 7498 and 161, the greater the number of dinucleotides lacking methylation indicating the more advanced progression to cervical cancer in the patient.

According to a third aspect of the present invention, there is provided a DNA primer having a sequence consisting of a sequence selected from the group consisting of sequence ID NOS.19, 20, 27 and 28.

According to a fourth aspect of the present invention, there is provided a kit for assessing the progression of cervical cancer in a patient infected with human papillomavirus comprising a methylation sensitive endonuclease and a pair of primers defining either end of an amplicon within the human papillomavirus genome, the amplicon containing at least one CpG site susceptible to cleavage by the methylation sensitive endonuclease.

Preferably, the primers are SEQ. ID NOS: 19 and 20.

Advantageously, the methylation sensitive endonuclease is McrBC endonuclease.

According to a fifth aspect of the present invention, there is provided a kit for assessing the progression of cervical cancer in a patient infected with human papillomavirus comprising:

an agent capable of selectively modifying the sequence of a CpG site depending upon the methylation status of the site; and

a pair of primers defining either end of an amplicon within the human papillomavirus genome, the amplicon containing at least one CpG site susceptible to modification by the agent.

Conveniently, the human papillomavirus is HPV-16.

Preferably, the pair of primers are SEQ. ID NOS. 27 and 28.

Advantageously, the kit further comprises at least one detection probe, the detection probe comprising a detectable marker and being capable of hybridising to said CpG site with a first melting temperature and being capable of hybridising to the site with a second, different melting temperature when the CpG site is modified.

Preferably, the human papillomavirus is HPV-16 and the at least one detection probe is capable of hybridising to the CpG site at the position in the HPV-16 genome selected from the group consisting of 7535, 7554, 7677, 7683, 7695, 7862, 31, 37, 43, 52 and 58.

Advantageously, there are eleven detection probes, the detection probes being capable of hybridising to the CpG site at positions 7535, 7554, 7677, 7683, 7695, 7862, 31, 37, 43, 52 and 58, respectively, in the HPV-16 genome.

Conveniently, the kit further comprises at least one anchor probe capable of hybridising to a position on the human papillomavirus genome adjacent the at least one detection probe, the anchor probe comprising a detectable marker, the detectable marker of the detection probe being selected from one of a donor dye and an acceptor dye, the detectable marker of the anchor probe being selected from the other of the donor dye and the acceptor dye, the donor and acceptor dyes being such that they are capable of fluorescence resonance energy transfer when they are in close proximity to one another.

Preferably, the donor dye is Fluorescein and the acceptor dye is selected from the group consisting of Light Cycler-Red 640 and Light Cycler-Red 705.

Advantageously, the detection probe is capable of hybridising to the CpG site at position 7862 in the HPV-16 genome.

Conveniently, the agent is selected from the group consisting of sodium bisulfite and potassium permanganate.

### BRIEF DESCRIPTION OF THE DRAWINGS

FIG. 1 is a diagrammatic representation of a portion of the HPV-16 genome. It shows the distribution of CpGs in the LCR (position 7154 through 7906/1 to 96) and in the E6 gene of HPV-16 (position 97 to 559) and the position of two Hpall/Mspl sites, one (position 57) overlapping with elements of the E6 promoter P97, the other one in the 3′ part of the E6 gene (position 502). Also shown are amplicons P11, P2 and P5 whose respective primers are shown in Table 1.

FIG. 2 is an image of an agarose gel showing the results of reverse transcription PCR confirming that similar amounts of E6 and E7 transcripts are generated by SiHa and CaSki cells. GAPDH (glyceraldehyde-3-phosphate dehydrogenase), served as cellular control transcript.

FIG. 3 is an image of an agarose gel showing the results of genomic PCR of SiHa HPV-16 DNA at I×, 10×, and 100× concentration compared with CaSki HPV-16 DNA at I× concentration. This confirms the large excess of HPV-16 DNA in CaSki cells.

FIG. 4 is an image of agarose gels following the analysis of the methylation status in the PCR amplicons P5 (corresponding to the E6 gene). P2 (containing the LCR) and P11 (containing just E6 promoter sequences). (The amplicons are shown schematically in FIG. 1). Chromosomal DNA of SiHa and CaSki cells was not cut (control, CT) or cleaved with either of the two enzymes (Hpall, H, and Mspl, M, respectively), and PCR amplified to generate the amplicons P2, P5 and P11. Each of the three amplicons could not be generated after cleavage with either of the two enzymes in the case of SiHa cells. This indicates the lack of any methylated CCGG sequences. In the case of CaSki cells, most of the DNA was resistant to Hpall digestion, but readily cleaved by Mspl, indicating methylation of these two sites in most of the 500 HPV-16 copies of CaSki.

FIG. 5 is a genomic map of HPV-16 (7906 bp) showing the genes E6, E7, El, E2, E5, L2, L1, and the long control region (LCR) and relative location of the amplicons G1 to G8. The respective primers of amplicons G1 to G8 are shown in Table 1.

FIG. 6 is a set of images of agarose gels showing segments of the HPV-16 genome following McrBC endonuclease digestion. Cleavage of the amplicons G1 to G8 by McrBC (right lane of each pair of samples) indicates methylation, compared with uncleaved controls. The cleavage pattern indicates hypermethylation throughout most HPV-16 genomes in DNA from CaSki cells and tumor 6. The cleavage pattern also indicates hypermethylation of the late genes in the single HPV-16 genome of SiHa, and hypo- or no methylation in tumor 4 and a CIN I lesion.

FIG. 7 is a diagrammatic representation of the amplicon P4 which includes the genomic segment from position 7850 to 559 with the E6 promoter elements, the E6 oncogene and cis-responsive elements within E6 (left side of figure). The primers of the P4 amplicon are shown in Table 1.

FIG. 8 is a pair of agarose gels of segments of HPV-16 genomes subjected to McrBC digestion (Mc), compared with controls (CT). The upper panel shows two typical McrBC resistant (i.e. unmethylated) P4 amplicons detected in DNA from invasive cancers. The lower panel shows two typical McrBC sensitive and methylated P4 amplicons from asymptomatic cervical smears.

### DESCRIPTION OF THE PREFERRED EMBODIMENTS

The data in the examples provide compelling evidence that the HPV-16 genome is efficiently modified by cellular DNA methylation, and show the role which DNA methylation has in the HPV-16 biology, and how methylation can be used to understand the etiology of and to diagnose cervical cancer.

HPV-16 DNA may not become recognized as a methylation target by a single physical property. It is not targeted only as an episome, since methylation was observed not only in asymptomatic smears containing presumably replicating HPV-16, but also in invasive tumors and cell lines, which typically contain HPV-16 genomes integrated into chromosomal DNA (zur Hausen, H.). Repeated DNA is thought to be a preferred target for CpG methylation (Yoder et al.), but in situ, monomeric HPV-16 episomes are methylated as is also the case in the cell line SiHa with regard to the non-transcribed part of the single HPV-16 genome. It has been proposed that recombination events provide a de novo methylation signal (Remus et al.), possibly corresponding to HPV-16 methylation in some tumors .

The meCpG mapping described in the Examples shows that the methylation of the HPV-16 LCR does not follow a singular and simple pattern, but that groups of CpGs in alternative locations and with alternative extent can become modified. Methylation of numerous CpGs of the enhancer indicates repression of its function. Since CpG methylation does not interfere with binding of the E6 promoter factor Sp1 (Harrington, M. A., et al.) a methylated promoter, however, may be transcriptionally active independent of this enhancer.

The normal HPV-16 lifecycle is restricted to asymptomatic infections and CIN lesions, while carcinogenic progression is an accident, as there is no further virus production. Consequently, the high prevalence of methylated HPV-16 genomes in asymptomatic epithelia may indicate that methylation is part of the normal HPV-16 biology. While not wishing to be bound by the exact mechanism, two speculative and opposing views might explain this observation.

The cell may have an antiviral defense that senses viral DNA as foreign and targets it for transcriptional repression. Alternatively, DNA methylation may be yet another example of the numerous strategies developed by HPVs that favor a subclinical, long term maintenance of the viral infection (Bernard et al.). Such a model would be reminiscent of the lifecycle of Epstein-Barr-Virus, which includes DNA methylation-dependent silencing of a specific promoter during one form of latency (Robertson, K. D.).

Likewise, the HPV genome may contain molecular properties that induce methylation dependent latency under some conditions, while under different conditions this mechanism may be overruled. This could be achieved by the conserved nuclear matrix attachment regions (Stünkel et al.) and Sp1 sites at the E6 promoters of all genital HPVs (Tan et al.). Both elements have been reported to antagonize DNA methylation in the context of certain cellular genes (Brandeis, M. et al. and Forrester, W. C., et al.). Preferential methylation of late genes may suggest an additional role in the early-late switch, which is a multifaceted event involving differential promoter usage, splicing, elongation, and mRNA stability (Baker, C. C. et al. and Ozbun, et al.).

However, irrespective of the molecular reasons for methylation of the HPV genome, the data in the examples show the power of molecular diagnoses by revealing the epigenetic factors which underly pathological samples.

Thus, the present invention relates, in general, to a method of diagnosing cervical cancer in a patient infected with human papillomavirus HPV-16. The method comprises providing a sample containing the HPV-16 genomic DNA contained in the cervical cells of the patient. Preferably, the sample is obtained from a cervical swab of the patient. The human papillomavirus DNA is, in some embodiments, the HPV genome which has integrated into the patient's cellular genome or, in other embodiments, the HPV genome which exists as an episome in the cervical cells of the patient. The method also comprises the step of determining the presence or absence of methylation of CpG sites in the HPV genome in the sample. The “CpG” sites in the genome are those consisting of a cytosine residue directly 5′ to a guanine residue. In different embodiments of the invention, the determination of the presence or absence of methylation is carried out in different ways, as is explained below.

The greater the absence of methylation of the CpG sites the more indicative of the presence of cervical cancer or, alternatively, the more indicative of the progression of the HPV infection to a transformed state, such as the presence of a precursor lesion or the presence of cervical lesions of increasing severity. Thus the lower the presence of methylated CpG sites the greater the indication of progression from subclinical infection to invasive cervical carcinoma. Conversely, the greater the presence of methylation of the CpG sites, the more indicative that the patient is asymptomatic.

Thus in some embodiments of the invention, the method of diagnosing cervical cancer does not provide a qualitative test for the presence of cervical cancer but rather provides a quantitative test for the progression of HPV infection to malignancy. It is also to be appreciated that, within this specification, the word “diagnosis” means not only the steps leading to an unequivocal identification of an ailment but also steps leading to contributory information which a physician can use when forming an opinion as to what an ailment may be.

The human papillomavirus with which the patient is infected is HPV-16. HPV-16 is defined as a human papillomavirus having a sequence corresponding to the sequence set out in the Los Angeles Laboratory Theoretical Biology and Biophysics Papillomavirus database, The enhancer promoter region of HPV-16 between the positions 7498 and 161 contains eleven CpG dinucleotides at the positions 7535, 7554, 7677, 7683, 7695, 7862, 31, 37, 43, 52 and 58. The enhancer promoter region of the HPV-16 includes the long control region (LCR) and promoter P97 of the E6 and E7 oncogenes in HPV. Therefore, the presence or absence of methylation at these eleven CpG dinucleotides is determined in order to indicate the existence or progression of cervical cancer. This is because it has been found that these particular CpG dinucleotides are particularly indicative of the status of HPV-16 infection towards malignancy.

Thus, in preferred embodiments, the presence of methylation at all of these eleven CpG dinucleotides indicates that there is no progression towards malignancy.

In preferred embodiments, the absence of methylation in at least one of the CpG dinucleotides at positions 37, 43, 52 and 58 of the HPV-16 genome indicates that the patient has at least a precursor lesion at stage CIN I (i.e. cervical intraepithelial neoplasia I). Thus it is indicative of either the presence of a CIN I precursor lesion, a CIN III precursor lesion or invasive cancer.

One of the eleven CpG dinucleotides between position 7498 and 161 of the HPV-16 genome is a CpG dinucleotide at position 7862. It has been found that the CpG dinucleotide at position 7862 is particularly informative as to the progression of HPV infection to malignancy in an individual. Consequently, in particularly preferred embodiments of the invention, the presence or absence of methylation at this CpG dinucleotide is determined specifically. In these embodiments, the absence of methylation at position 7862 indicates that the patient has at least a precursor lesion at stage CIN I. Thus it indicates that the patient either has a precursor lesion at stage CIN I or a precursor lesion at stage CIN III or has invasive cancer.

As was explained above, the presence or absence of methylation of the CpG genome is determined in different ways in different embodiments of the invention.

In one embodiment, the presence or absence of methylation is determined by digesting the HPV genome with the McrBC endonuclease. The McrBC endonuclease is a methylation-sensitive endonuclease which cuts close to the sequence purine-meC in the context of a second, arbitrarily spaced purine-meC. On average, the McrBC endonuclease cleaves the genome at every other methylated CpG dinucleotide. However, it is to be understood that the use of McrBC is not essential to the invention and in alternative embodiments, a different methylation-sensitive endonuclease is used instead of McrBC.

After digestion by McrBC, the digested genome is subjected to the PCR process using a pair of primers. In accordance with the PCR process, the primers define either end of an “amplicon”, i.e. the region of the genome which is actually amplified by the PCR process. The primers are generated so that they define an amplicon which includes at least one CpG site which is susceptible to digestion by the McrBC endonuclease, when methylated. Thus if the CpG site was, indeed, methylated and thus cleaved by the McrBC endonuclease then the PCR process will, in fact, be largely unsuccessful because the amplicon region of the genome is cleaved into at least two pieces (depending upon the number of CpG sites in the amplicon) preventing synthesis of the full amplicon strand. This also prevents subsequent amplification of that strand in the next round of the PCR process. If, on the other hand, no CpG sites which were susceptible to McrBC endonuclease digestion were methylated then the amplicon is not cleaved by the endonuclease and the PCR process amplifies the amplicon as normal.

In preferred embodiments, the primers are selected from SEQ. ID NOS. 1 to 24. In particularly preferred embodiments of the invention, the primers of SEQ. ID NOS. 19, and 20 are used because these two primers are complementary to sequences at either end of the E6 gene of HPV-16. Thus the primers define either end of the amplicon which has been shown to include CpG sites whose methylation is indicative of the presence or advancement of cervical cancer.

The products resulting from the PCR process are then analysed, for example by separation on an agarose gel, to determine whether or not the amplicon has been amplified. If it has been amplified then the amplicon was not cleaved which indicates that these were not any methylated CpG sites in the amplicon which were cleavable by the McrBC endonuclease. Conversely, if the amplicon has not been amplified then it has been cleaved which indicates that there was at least one CpG site in the amplicon which was methylated.

In another embodiment of the present invention, the presence or absence of methylation is determined by adding sodium bisulfite to the sample. Sodium bisulfite has the effect of selectively modifying unmethylated cytosine residues to become uracil residues. Sodium bisulfite does not modify methylated cytosine residues. In this embodiment, after addition of sodium bisulfite, the HPV genome is amplified using primers specific for positions at either end of a sequence of the genome that contains CpG sites. The resulting amplicon is then sequenced using the same primers. The presence of a uracil residue in place of a cytosine residue which is part of a CpG dinucleotide indicates that the CpG dinucleotide was unmethylated whereas the presence of cytosine indicates that the CpG dinucleotide was methylated. This embodiment has the advantage over the McrBC digestion protocol that all CpG dinucleotides in the HPV genome can be analyzed for methylation. In preferred embodiments, the primers used are those of SEQ. ID. NOS: 27 and 28. These primers produce an amplicon containing the eleven CpG sites between positions 7498 and 161 of the HPV-16 genome.

In a further embodiment of the present invention, the step of determining the presence or absence of methylation of the HPV genome also requires the addition of sodium bisulfite in order to modify selectively unmethylated cytosine residues to uracil residues. However, in this embodiment single nucleotide polymorphism (SNP) detection is carried out, preferably using the LightCycler™ system (Roche Molecular Biochemicals) to detect the methylation state of the CpG sites.

In these embodiments, a pair of primers is used to carry out the PCR process on a region of the HPV genome, which region includes at least one CpG site. A detection probe is added to the sample of the HPV genome and primers, the detection probe being of a sequence complementary to a target including a sequence of CpG site and the surrounding nucleotides. The detection probe has attached to it a donor dye (such as fluorescein) at its 3′ end. The donor dye absorbs light of a first wavelength. The sequence of the detection probe is generated such that the melting temperature of the probe-target hybrid is different when the CpG site includes a cytosine residue compared with when the cytosine residue has been modified to a uracil residue by sodium bisulfite. Also added to the sample is an anchor probe which has a sequence complementary to a section of the HPV genome adjacent the target sequence to which the detection probe is complementary. In particular, the anchor probe is complementary to a sequence downstream of the detection probe such that, when hybridised, the probes are between one and five nucleotides apart. The anchor probe has an acceptor dye (such as Light Cycler-Red 640 or Light Cycler-Red 705) attached to its 5′ end. The acceptor dye emits light at a second wavelength, different from the first under the circumstances described below.

In use, amplification of the amplicon in the HPV genome defined by the two primers is completed by the PCR process. The temperature of the sample is then slowly raised and the sample is exposed to a light at the first wavelength. If the detection probe and the anchor probe are both hybridised to the amplicon then their respective donor dye and acceptor dye are brought into close proximity such that fluorescence resonance energy transfer occurs between the two dyes. Thus when the donor dye absorbs the light at the first wavelength, it is excited and part of the excitation energy is transferred to the acceptor dye. The acceptor dye subsequently emits light at the second, different wavelength. If, on the other hand, the probes are not hybridised to the target, or if only one probe is hybridised to the target then the acceptor and donor dyes are not spatially close enough together for fluorescence resonance energy transfer to occur and thus, although the light at the first wavelength is absorbed, there is no emission of light at the second wavelength.

The emission of light at the second wavelength from the sample is measured as the temperature of the sample is steadily increased. The melting temperature of the detection probe is different depending upon whether or not the CpG site contains a uracil residue instead of a cytosine residue. Thus, as the temperature of the sample is increased, the detection probe will disassociate from the target sequence at a different temperature depending upon whether or not the CpG site contains a uracil residue. When the detection probe disassociates from the target, the distance between the donor dye and the acceptor dye is too great for fluorescence resonance energy transfer to take place and thus the emission of light from the acceptor dye ceases. Thus when light at the second wavelength is no longer detected, it can accurately be determined that the melting temperature of the detection has been reached and thus whether or not the CpG site contains a uracil residue instead of a cytosine residue.

In preferred embodiments, a cervical cell sample is divided into eleven sub-samples, to each of which is added a detection probe and a corresponding anchor probe. The detection probe added to each sub-sample is of a different sequence, each detection probe being complementary to one of the eleven CpG sites between position 7498 and 161 of the HPV-genome. The anchor probes have sequences complementary to corresponding positions within the HPV genome. Thus each sub-sample can be monitored, as described above, to determine the methylation status of that particular CpG site.

In an alternative embodiment, the sample is not divided into sub-samples but instead, the eleven different detection probes and their corresponding anchor probes are added to the same sample. However, in this embodiment, each acceptor dye emits light at a different wavelength, each of which is detected separately. Thus it is possible to monitor the disassociation of each separate detection probe simultaneously in a single sample.

The advantage of these embodiments is that it is significantly faster to determine the methylation status of a particular CpG site than is possible when it is necessary to sequence the DNA.

It is to be appreciated that the use of sodium bisulfite to modify cytosine residues selectively depending on the methylation status of their respective CpG site is not essential to these embodiments of the present invention. In alternative embodiments, another agent capable of selectively modifying the sequence of a CpG site depending upon the methylation status of the site is used. In one embodiment, potassium permanganate is used which converts methylated cytosine residues to tymine residues. The use of potassium permanganate requires the use of different primer and probe sequences for single nucleotide polymorphism detection than those described in relation to sodium bisulfite modification but, in other respects, the method involved is substantially the same.

It is also to be appreciated that the term “selectively modifying” also includes the situations in which, although modification is attempted, no CpG sites are actually modified. For example, if sodium bisulfite is added but all cytosine residues at CpG sites are methylated then no modification of nucleotides will occur.

According to another aspect of the present invention, DNA primers are provided in order to carry out the PCR amplification steps described above. The primers are generated such that the amplicon which they define contains at least one CpG site. It is preferred that the amplicon includes at least one of the CpG sites between positions 7498 and 161 of the HPV-16 genome. The DNA primers consist of the sequences set out in SEQ. ID NOS. 1 to 24. It is preferred that the primers used are SEQ. ID NOS. 19 and 20, in particular, where methylation status is determined by. McrBC digestion.

In embodiments in which the methylation status of a sequence of the HPV-16 genome is determined by attempting to digest the sequence with a methylation sensitive endonuclease, it is important that the sequence includes at least one CpG site which is susceptible to cleavage by the endonuclease. This can be determine by carrying out an appropriate test assay using a proposed sequence and the endonuclease and determining if cleavage occurs when the CpG sites in the sequence are methtylated and unmethylated. For example, in embodiments where the endonuclease is McrBC, the protocol provided in Example 4 can be used.

In embodiments in which the methylation status of a sequence of the HPV-16 genome is determined by selectively modifying the sequence of a CpG site depending upon the methylation status of the site using an appropriate agent, it is important that the sequence includes at least one CpG site which is susceptible to modification by the agent. This can be determined by carrying out an appropriate test assay using a proposed sequence and the agent and determining if selective modification occurs when the CpG sites in the sequence are methylated and unmethylated. For example, in embodiments where the agent is sodium bisulfite, the protocol provided in Example 6 can be used.

In embodiments in which there is sodium bisulfite modification of the HPV genome, it is preferred that the primers are SEQ. ID NOS 27 and 28. These sequences correspond to position 7498 to 7522 and 161 to 140, respectively and span a part of the enhancer-promoter or in HPV-16.

In other embodiments of the present invention, kits are provided in order to detect the methylation state of the HPV-16 genome in the cervical cells of an individual suspected of having cervical cancer. Accordingly, in one embodiment, the kit comprises a methylation-sensitive endonuclease, such as McrBC and DNA primers as described above as being suitable for PCR amplification following McrBC digestion. In preferred embodiments, the kit is for detecting the presence or progression of cervical cancer in an individual infected with HPV-16 and the primers are SEQ. ID NOS. 19 and 20.

In an alternative embodiment, the kit comprises: an agent that is capable of selectively modifying the sequence of a CpG site depending upon the methylation status of the site; and DNA primers as described above as being suitable for PCR amplification following McrBC digestion. Thus the agent will modify a nucleotide (usually the cytosine nucleotide) of the CpG site depending upon whether or not the CpG site is methylated. For example in some embodiments, sodium bisulfite is provide which modifies unmethylated cytosine residues to uracil residues while leaving methylated cytosine residues unmodified. In other embodiments, potassium permanganate is provided which modifies methylated cytosine residues to tymine residues while leaving unmethylated cytosine residues unmodified. In preferred embodiments, the primers are SEQ. ID NOS. 27 and 28.

In preferred embodiments, the kit also comprises a detection probe and an anchor probe for a particular CpG site as is described above. The detection probe has one of an acceptor and a donor dye attached to it and the anchor probe has the other of the acceptor and donor dye attached to it. In a particularly preferred embodiment, the kit comprises a plurality of detection probes, each detection probe being complementary to a different CpG site in the HPV-16 genome. For example in some embodiments the detection probes are complementary to the eleven CpG sites between positions 7498 and 161 of the HPV-16 genome. A corresponding anchor probe is also provided for each detection probe.

The kits are used to provide diagnoses of cervical cancer in a readily accessible fashion.

### EXAMPLES

The following examples provide further exemplification of the invention but are not intended to limit the invention in any way.

### Example 1

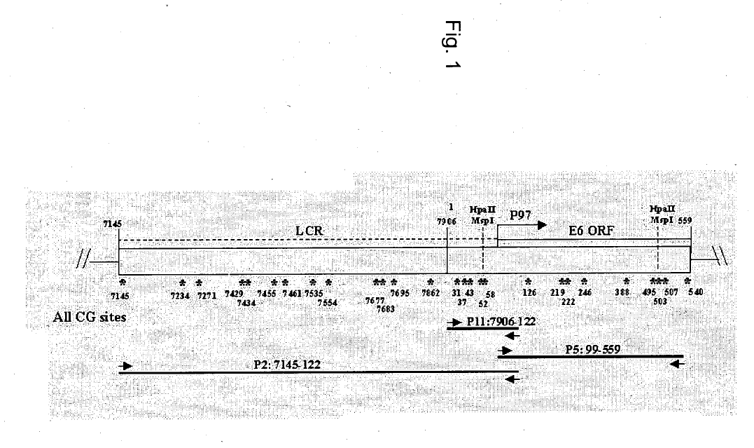

MeCpGs (i.e. methylated CpGs) can be detected with the restriction enzymes Hpall and Mspl. Both enzymes cleave the sequence CCGG, but CpG methylation inhibits cleavage by Hpall whereas Msp1 is unaffected by methylation. By carrying out digestions of the HPV-16 genome with Hpall and Mspl and sequencing the resulting fragments, a map showing the position of two Hpall/Mspl sites in the LCR and E6 gene of HPV-16 was produced and this is shown in FIG. 1.

In view of their genome sizes of 7900 bp and G+C contents close to 40%, one might expect to find in HPV genomes about 400 CpG sites. However, the common papillomavirus types, HPV-6, 11, 16, 18, 31 and 45 have 160, 155, 112, 172, 122, and 154 CpGs, respectively (Myers, G. et al.). This lower than expected frequency, similar to the human host, suggests a function of CpGs in the biology of genital HPVs.

### Example 2

The cervical carcinoma cell lines CaSki and SiHa contain about 500 and a single genome, respectively, of HPV-16, integrated into the chromosomal DNA. The HPV-16 genome of SiHa is integrated by interruption of its E2 gene at the positions 3132/3384, accompanied by deletion of 251 nucleotides of viral sequence (Baker et al.).

The HPV-16 genomes in CaSki are inserted in numerous loci. In most of these, HPV-16 is transcriptionally silent, while E6/E7 transcripts stem from a single locus (Remus, R. et al.), possibly due to an epigenetic repression mechanism.

CaSki and SiHa cells originated from H. zur Hausen at the German Cancer Research Center, Heidelberg, where they were frozen in 1986 and freshly thawed for this study. Cellular DNA was purified with Qiagen genomic tips. 15 and 150 ng of each of these DNAs, respectively, were digested with Hpall and Mspl (New England Biolabs), and after heat inactivation of the enzymes amplified by PCR.

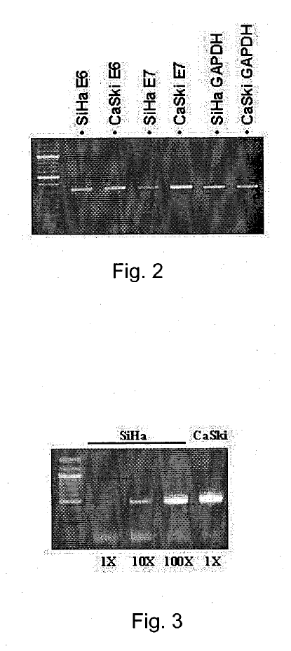

Reverse transcription PCR of E6, E7 and GAPDH genes was carried out and this was visualised on an agarose gel which is shown in FIG. 2. For comparative purposes, genomic PCR of SiHa HPV-16 DNA at I×, I0× and I00× concentration and of CaSki HPV-16 DNA at I× concentration were also carried out. The results were visualised on an agarose gel which is shown in FIG. 3.

As can be seen in FIG. 2, SiHa and CaSki cells produced approximately similar quantities of E6 and E7 transcripts whereas FIG. 3 shows that CaSki cells have considerably higher levels of HPV-16 DNA than SiHa cells. Therefore, this example confirmed that SiHa and CaSki cells express similar levels of E6 and E7 transcripts, in spite of the large excess of HPV-16 genomes in CaSki cells (Baker et al.).

### Example 3

To inquire whether CpG methylation may explain the observation of Example 2, the methylation status was analyzed of the two Hpall/Mspl sites in the LCR and E6 gene. These sites are present in the PCR amplicons P5 (corresponding to the E6 gene), P2 (containing the LCR), and P11 (containing just E6 promoter sequences) in both DNAs. The positions of the P5, P2 and P11 amplicons are shown schematically in FIG. 1. As can be seen, these amplicons were used to study the enhancer-promoter E6 segment specifically.

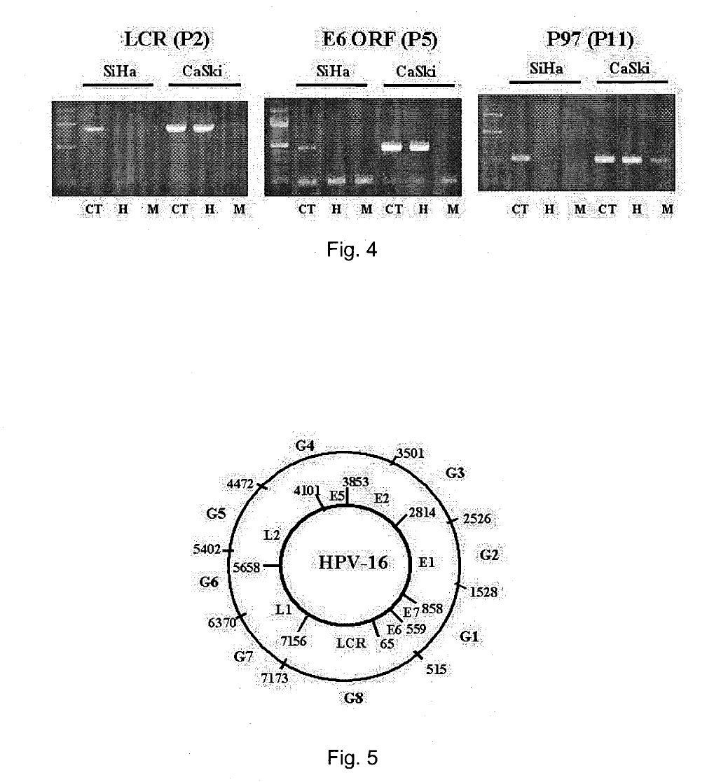

Chromosomal DNA of SiHa and Caski cells was separated into samples which were either not cut (control, CT) or were cleaved with one of the two enzymes (Hpall, H, and Mspl, M, respectively). The samples were then amplified using the PCR process to generate the amplicons P2, P5 and P11. The respective primers are shown in Table 1. The PCR protocol is set out below. The resulting samples were separated on an agarose gel which was visualised and is shown in FIG. 4.

In the case of SiHa DNA, these amplicons could not be generated after cleavage with either of the two enzymes, while in the case of CaSki cells, most of the DNA was resistant to Hpall, but readily cleaved by Mspl.

These findings indicate that the LCR and the E6 gene contain CmeCGG in most HPV-16 genomes of CaSki but not in the HPV-16 genome of SiHa cells. Thus most HPV-16 genomes of the cancer cell line CaSki contain meCpGs, but not the single HPV-16 genome of SiHa cells.

PCR was carried out in 25 μl containing 0.2 mM of each of the four dNTPs, 10 pmol primers, 2.5 μl of buffer B supplied by the producer (Promega), 2 mM MgCl₂ and 0.75 units Taq (Promega) with 25 or 0.25 μl of SiHa and CaSki DNA, respectively, uncleaved or cleaved by McrBC. The PCR started at 94° C. for 1 min followed by 35 amplification cycles (denaturation 94° C., 10 sec, annealing 58° C., 30 sec, extension 68° C., 45 sec increasing by 10 sec per cycle), final extension at 68° C., 7 min. PCR with Taq Gold was carried out in 25 μl containing template DNA, 5 mM dNTPs, 10 pmol primers, 2.5 μl of magnesium ion free buffer supplied by the manufacturer, 2mM MgCl₂ and 1.25 units AmpliTaqGold (Applied Biosystems) at 94° C., 9 min followed by 40 amplification cycles (denaturation at 94° C., 10 sec, annealing 58° C., 30 sec, extension 68° C., 45 sec increasing by 10 sec per cycle), final extension at 68° C., 7 min.

TABLE 1

| | | | | |
|---|---|---|---|---|
| Amplicon | Primer | Position | Sequence | SEQ ID NO: |

| | | | | |
|---|---|---|---|---|
| G1 | G1F | 515-534 | GTCTTGTTGCAGATCATCAAGA | 1 |
| | G1R | 1528-1509 | ATTCTGAAAAACTCACCCCG | 2 |
| G2 | G2F | 1501-1520 | GAGTTATACGGGGTGAGTTT | 3 |
| | G2R | 2526-2505 | CAATGGTCTATGCTTTACATCC | 4 |
| G3 | G3F | 2501-2522 | CTATGGATGTAAAGCATAGACC | 5 |
| | G3R | 3501-3481 | TTTCCGGTGTCTGGCTCTGAT | 6 |
| G4 | G4F | 3471-3490 | AGCGACCAAGATCAGAGCCA | 7 |
| | G4R | 4472-4453 | GGCCTTGTTCCCAATGGAAT | 8 |
| G5 | G5F | 4402-4421 | GGTGGGTTAGGAATTGGAAC | 9 |
| | G5R | 5402-5383 | GATGTAGAGGGTACAGATGG | 10 |
| G6 | G6F | 5367-5386 | TTCTACAACCCCGGTACCAT | 11 |
| | G6R | 6370-6351 | GTCGCCATATGGTTCTGACA | 12 |
| G7 | G7F | 6348-6367 | TGGTGTCAGAACCATATGGC | 13 |
| | G7R | 7173-7150 | CAACATACATACAATACTTACAGC | 14 |
| G8 | G8F | 7145-7184 | CGTAAGCTGTAAGTATTGTATGTA | 15 |
| | | | TGTTGAATTAGTGTTG | |
| | G8R | 559-528 | TTACAGCTGGGTTTCTCTACGTGT | 16 |
| | | | TCTTGATG | |
| P2 | P1F | 7145-7184 | CGTAAGCTGTAAGTATTGTATGTA | 17 |
| | | | TGTTGAATTAGTGTTG | |
| | P2R | 122-94 | CCTGTGGGTCCTGAAACATTGCAG | 18 |
| | | | TTCT | |
| P4 | P4F | 7851-7886 | TGTGTGCAAACCGTTTTGGGTTAC | 19 |
| | | | ACATTTACAAG | |
| | P1R | 559-528 | TTACAGCTGGGTTTCTCTACGTGT | 20 |
| | | | TCTTGATG | |
| P5 | P5F | 99-132 | ATGCACCAAAAGAGAACTGCAAT | 21 |
| | | | GTTTCAGGAC | |
| | P5R | 559-528 | TTACAGCTGGGTTTCTCTACGTGT | 22 |
| | | | TCTTGATG | |
| P11 | P11F | 1-38 | ACTACAATAATTCATGTATAAAAC | 23 |
| | | | TAAGGGCGTAACCG | |
| | P2R | 122-94 | CCTGTGGGTCCTGAAACATTGCAG | 24 |
| | | | TTCT | |

### Example 4

A screen with the restriction enzyme McrBC is more powerful than the analysis by Hpall/Mspl, as the McrBC enzyme cuts close to the sequence purine-meC (PumeC) in the context of a second, arbitrarily spaced PumeC (Sutherland, E., et al. and Stewart, F. J. et al.). As a consequence, McrBC recognizes pairs of PumeCpG residues, and, on the average, cleaves every other meCpG, while Hpall/Mspl analysis resolves only the methylation status of one in 16 CpGs.

The HPV-16 genome contains 112 CpGs, and 81 of these are part of the sequence purine-CpG, thus being potential targets of methylation as well as for cleavage by McrBC. To establish a crude map of the distribution of methylation throughout the HPV-16 genomes of SiHa and CaSki cells, the chromosomal DNA was digested with McrBC, and segments with sizes of about 1 kb were amplified using the PCR process.

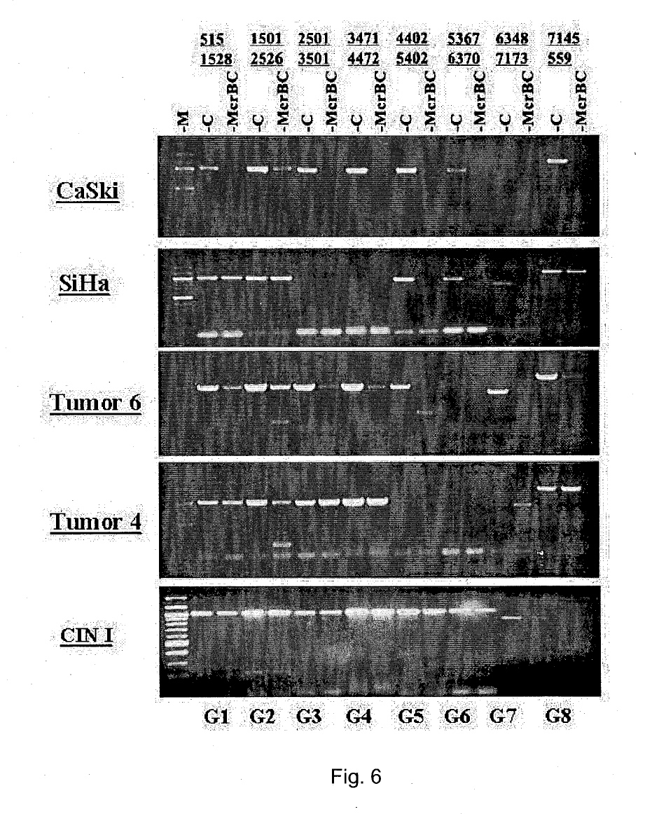

The HPV-16 genome and the respective amplicons G1 to G8 generated by the PCR process are shown schematically in FIG. 5. These amplicons overlap and comprehensively cover the whole HPV-16 genome. The primers defining the ends of each amplicon are shown in Table 1. After the PCR process, the samples were separated on an agarose gel and the results are shown in FIG. 6.

The PCR protocol used was the same as in Example 3 except that for digestions with McrBC (New England Biolabs), 250 ng of SiHa or CaSki DNA were digested with 3 units of enzyme for 1 hr at 37° C. in 25 μl NE buffer 2 (50 mM NaCl, 10 mM Tris-HCl, 10 mM MgCl₂, 1 mM dithiothreitol pH 7.9).

As expected from the Hpall/Mspl data, the LCR-E6 segment from position 7145 to 559 could not be digested in SiHa DNA, indicating a lack of methylated targets. Most of this amplicon was digested in CaSki DNA and therefore methylated, while a reproducible weak undigested band indicated lack of methylation of some copies. Also, the segments 515-1528 and 1501-2526 were not cleaved in SiHa and only partially cleaved in CaSki, suggesting a lack of and partial methylation of, respectively, the genomic region spanning the E7 and E1 genes. The amplicon between the position 2501-3501 could not be amplified with SiHa DNA as expected from the recombination of this segment. Most of this amplicon was digestible with CaSki DNA. Three of the remaining segments (3471-4472, 5367-6370, and 6348-7143) were almost completely cleaved with both SiHa and CaSki DNA, while a fourth segment (4402-5402) was partially cleaved. This part of the genome with the late genes L2 and L1 is apparently well recognized by the cellular methylation machinery. In SiHa this segment is—due to the recombination—upstream of viral promoters and may be transcripionally silent.

In order to examine whether one would find a similar distribution of McrBC cleavage in situ, the DNA from a cervical carcinoma (tumor 6 which corresponds to “T6” referred in Example 7 and Table 3) was analyzed. This was the only DNA preparation among 33 tumor derived DNAs, which contained a strongly methylated LCR-E6 segment (see below). This tumor DNA was preferentially cleaved in a similar uneven manner as the HPV-16 genomes in SiHa and CaSki cells with strong methylation of all segments between positions 3471 and 7173. The 7145-557 and the 2501-3501 segments were moderately methylated, while there was low methylation in the segment from 515 to 2526 (see FIG. 6). Differential cleavage by McrBC was not biased by unequal distribution of McrBC sites, since the amplicons G1 to G8 have all numerous potential McrBC sites (11, 8, 15, 13, 5, 7, 10, and 13 respectively). For example, G6 with seven sites was efficiently cleaved in CaSki, SiHa and T6 DNA, in contrast to G2, which has eight sites.

The DNA from a carcinoma, whose LCR-E6 segment had, like those of nearly all tumors studied, no detectable DNA methylation (see below) was also examined. The DNA of tumor 4 was not cleaved by McrBC throughout the HPV-16 genome (see FIG. 6). The segment 5367-6370 led only to a weak amplicon.

The complete HPV-16 genome of a precursor lesion (cervical intraepithelial neoplasia I, CIN I) was also analyzed (see FIG. 6). None of the amplicons of this sample, initially found to have a hypo-methylated LCR-E6 region, was cleaved by McrBC.

This example shows that HPV-16 DNA can be extensively modified by DNA methylation. Methylation varies quantitatively among different parts of the genome, and hypermethylation correlates in three DNA isolates with the position of the late genes. Methylation of HPV-16 DNA is not necessary but depends on yet unknown circumstances as suggested by the lack of detectable methylation in two lesions, the tumor likely containing integrated DNA, the CIN I lesion episomal DNA.

### Example 5

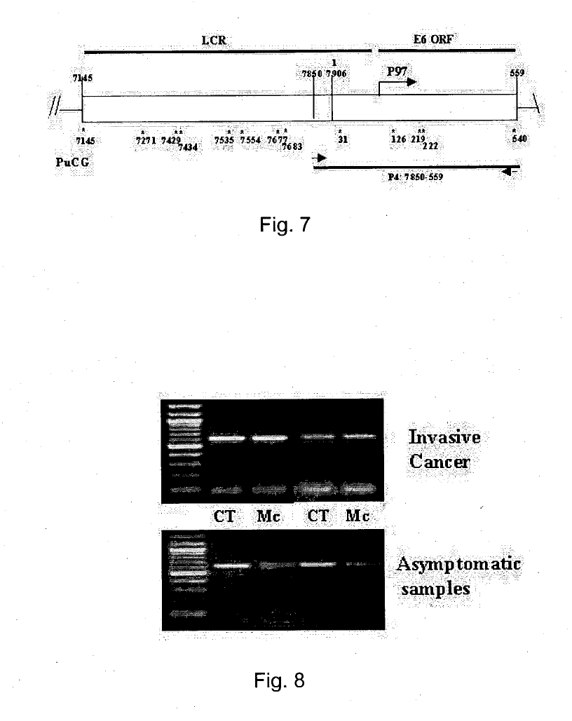

In order to determine whether CpG methylation occurs only sporadically in cell lines and in invasive carcinomas or whether it is frequent in clinical specimens, the following experiments were carried out. DNA from cervical smears classified as normal (“asymptomatic smears”), biopsies of low-grade and high-grade precursor lesions (CIN I and CIN III) and invasive carcinomas were analyzed by McrBC digestion and PCR amplification using the P4F and P1R primes shown in Table 1. The strategy is shown in FIG. 7. The results of PCR amplification were then analyzed by separation on an agarose gel, some of which are shown in FIG. 8. As can be seen in FIG. 8, bands on the gels of samples from individuals with invasive cancer were unaffected by digestion with McrBC. However bands on the gel of asymptomistic samples were fainter than digested with McrBC.

DNA from 48 clinical specimens from New Mexico and 33 from Brazil were examined.

Brazilian samples (L.L.V.) originated from cross-sectional studies in two cities in the Northeast of the country, and consisted of cervical scrapings and tumor biopsies. Cytological and histopathological analyses followed the Bethesda system. For smears classified as “asymptomatic”, ectocervical and endocervical cells were collected with a cytobrush. Tissue biopsies were classified as cervical intraepithelial lesions (CIN I and III) or invasive carcinoma, and digested with proteinase K prior to DNA isolation. DNA samples were purified by spin column chromatography and tested for the presence of HPV DNA by the MY09/11 PCR protocol for specific HPV types (Bernard, H. U. et al.). All specimens were number coded for privacy of the subjects. Asymptomatic and precursor samples from New Mexico, USA, were obtained and characterized during published epidemiological research (Peyton C. L. et al.). Invasive cervical cancer specimens were obtained from ongoing case-control studies of the same population diagnosed between 1980 and 1999 (C.M.W.). The results are shown in table 2.

The upper panel of table 2 describes the composition and analysis of each cohort. CpG methylation was determined by cleavage of the DNA preparations with the restriction enzyme McrBC and subsequent PCR amplification of a genomic segment of HPV-16 corresponding to the promoter and the E6 gene as shown in FIG. 7. The lower panel of table 2 was generated by uniting the data from both cohorts as well as CIN I and III into one group of precursor lesions, and omitting DNA preparations that could not be amplified.

Among the Brazilian specimens, 8 of 14 asymptomatic smears contained hypermethylated viral DNA, none of 3 CIN I lesions, and 2 of 16 samples from carcinomas. One of these two carcinoma DNAs was lightly methylated, the other one, tumor 6, strongly, and was described in detail above. Among the New Mexican samples, DNA of 5 of 11 asymptomatic smears contained methylated HPV-16 genomes, 3 of 10 CIN I lesions, 2 of 10 CIN III lesions, and none of 17 cancers. Taking both cohorts together, 52% of the HPV-16 genomes from asymptomatic smears contained methylated DNA, 21.7% of precursor lesions, and only 6.1% of the carcinomas, indicating that methylation of the LCR and the E6 gene decreases with progression of the infection.

A scan of the methylation pattern of the whole HPV-16 genome from cervical smears of asymptomatic patients could not be attempted because of the small amounts of DNA available.

This Example shows that HPV-16 methylation is frequent in asymptomatic smears, uncommon in precursors lesions, and rare in carcinomas.

TABLE 2

| | | | |
|---|---|---|---|
| | | | % Methylation of |
| Methylated | Unmethylated | Total | amplifiable samples |

Brazil

| | | | | |
|---|---|---|---|---|
| Asympto- | 8 | 6 | 14 | 57.1 |
| matic | | | | |
| samples | | | | |
| CIN I | 0 | 3 | 3 | 0 |
| Invasive | 2 | 14 | 16 | 12.5 |

New Mexico USA

| | | | | |
|---|---|---|---|---|
| Asympto- | 5 | 6 | 11 | 45.4 |
| matic | | | | |
| samples | | | | |
| CIN I | 3 | 7 | 10 | 30 |
| CIN III | 2 | 8 | 10 | 20 |
| Invasive | 0 | 17 | 17 | 0 |

Brazil + New Mexico

| | | | | |
|---|---|---|---|---|
| Asympto | 13 | 12 | 25 | 52 |
| matic smears | | | | |
| CIN I + III | 5 | 18 | 23 | 21.7 |
| Invasive | 2 | 31 | 33 | 6.1 |
| Cancer | | | | |

### Example 6

meCpGs can be mapped precisely by sequencing after sodium bisulfite modification (Van Tine, B. A., et al.,). Sodium bisulfite has the effect of modifying unmethylated cytosine residues to become uracil residues. As it is too laborious to analyze completely 7.9 kb genomes in numerous samples, this technique was used only for selected samples and an analysis of the enhancer-promoter region between the genomic positions 7498 and 161. This region contains eleven CpGs at the positions 7535, 7554, 7677, 7683, 7695, 7862, 31, 37, 43, 52, and 58 (see FIG. 1). The first five of these CpGs are in close proximity to important AP1 and NFI sites of the HPV-16 enhancer (Bernard et al. Apt, D et al., and Gloss, B et al.). The last five CpGs overlap with an Sp1 and two E2 binding sites, which regulate the transcription start at position 97 (Demeret. C et al. and Apt. D et al.).

For mapping of methylated cytosine residues, DNAs were modified by the CpGenome™ DNA modification kit of Intergen Inc., and the reaction products amplified with primers specific for modified HPV-16 DNA. Msp3F (genomic position 4322-4348, ATTTGATATTATATTTAAGGTTGAA SEQ. ID NO: 25) and msp3R (4970-4946, AATAATTACAAAAACAAAATCTACA SEQ. ID NO: 26) spanned part of the L2 gene and were used as controls. Msp4F (7498-7522, TAGTTTTATGTTAGTAATTATGGTT SEQ. ID NO: 27) and msp4R (161-140, ACAACTCTATACATAACTATAATA SEQ. ID NO: 28) amplified a segment with the enhancer-promoter. The amplification products were directly sequenced with the same primers.

In SiHa, all eleven of the CpGs were modified, i.e. unmethylated. In contrast, the same eleven CpGs between positions 7498 and 161 were methylated in most of the 500 HPV-16 DNA copies of CaSki cells. An unmodified and transcriptionally active background of one or a few HPV-16 genomes would not have been detectable in this experiment. The segment of the L2 gene defined by the Msp3F and Msp3R primers (4322-4946) served as a control and did not differ in SiHa and CaSki, as all four CpGs of this amplicon were methylated.

### Example 7

Fifteen patient samples were studied by bisulfite modification using the same protocol as in Example 6. The results are shown in Table 3. In the table, “As” refers to a sample from a patient who was asymptomatic; “CIN” refers to a sample from a patient having a precursor lesion of that type; and “T” refers to a sample from a patient having a cervical cancer tumour (i.e. invasive cancer). Key: +, methylation, −, lack of methylation, −(+) methylated as well as unmethylated residues in the same sample. The five CpGs between position 7535 and 7695 are close to cis-responsive elements of the viral enhancer, the CpGs between 31 and 58 overlap with promoter elements. The CpG 7862 is between the borders of two specifically positioned nucleosomes overlapping the enhancer and promoter, respectively.

In five asymptomatic smears, all eleven CpGs were methylated. In two CIN I samples, three and four, respectively, of the five CpGs at the promoter were not methylated, while all CpGs of the enhancer were methylated in one sample and two in the other one. A CIN III sample and two tumor samples were methylated at all CpGs of the enhancer and promoter, while five other tumors had heterogenous methylation patterns. Notably, the only CpG residue between the enhancer and promoter (position 7862) was unmethylated in all ten CIN and invasive cancer lesions. This site is the only CpG dinucleotide between two specifically positioned nucleosomes and close to an AP-1 site essential for transcriptional activation during epithelial differentiation (Bernard, H. U. et al. and O'Connor M. J. et al.). The same site was also unmethylated in six other tumors, most of which had some additional unmethylated CpGs in the enhancer or promoter segment.

The present invention has been exemplified by the above described embodiments. However, those skilled in the art will appreciate that further embodiments can be provided which are also within the spirit and scope of the invention.

TABLE 3

| Position | 7535 | 7554 | 7677 | 7683 | 7695 | 7682 | 31 | 37 | 43 | 52 | 58 |
|---|---|---|---|---|---|---|---|---|---|---|---|
| SiHa | − | − | − | − | − | − | − | − | − | − | − |
| SaSki | + | + | + | + | + | + | + | + | + | + | + |
| As16 | + | + | + | + | + | + | + | + | + | + | + |
| As22 | + | + | + | + | + | + | + | + | + | + | + |
| As23 | + | + | + | + | + | + | + | + | + | + | + |
| As24 | + | + | + | + | + | + | + | + | + | + | + |
| As33 | + | + | + | + | + | + | + | + | + | + | + |
| ClNIA1 | + | + | + | + | + | − | + | − | − | − | − |
| ClNIA6 | + | + | − | − | − | − | + | + | − | − | − |
| ClNIIIA20 | + | + | + | + | + | − | + | + | + | + | + |
| T6 | + | + | + | + | + | − | + | + | + | + | + |
| T8 | + | −(+) | − | + | − | − | − | − | + | + | + |
| T9 | ? | + | + | −(+) | −(+) | −(+) | + | + | + | + | + |
| T10 | + | + | + | + | + | − | + | + | + | + | + |
| T20 | ? | ? | + | −(+) | −(+) | −(+) | + | + | + | + | + |
| T37 | − | − | − | − | − | − | + | + | + | + | + |
| T39 | + | + | + | + | + | − | − | − | − | − | − |

### REFERENCES

Apt, D., Chong, T., Liu, Y. & Bernard, H. U. Nuclear factor I and epithelial cell-specific transcription of human papillomavirus type 16. J. Virol. 67: 4455-4463 (1993).

Baker, C. C. et al. Structural and transcriptional analysis of human papillomavirus type 16 sequences in cervical carcinoma cell lines. J. Virol. 61, 962-971 (1987).

Baker, C. C. & Calef, C. Maps of papillomavirus transcripts. In Human papillomaviruses Compendium (eds. Myers, G. et al.) III-3-III-20 (Los Alamos National Laboratory, Los Alamos, New Mexico, USA, 1995).

Bernard, H. U. Regulation of human papillomavirus gene expression by the factor CDP, nucleosomes and the nuclear matrix. Papillomavirus Report 11,73-80 (2000).

Bernard, H. U. et al. Assessment of known and novel human papillomaviruses by polymerase chain reaction, restriction digests, nucleotide sequence, and phylogenetic algorithms. J. Inf. Dis. 170, 1077-1085 (1994).

Bird, A. P. The essentials of DNA methylation. Cell 70: 5-8 (1992).

Brandeis, M. et al. Sp1 elements protect a CpG island from de novo methylation. Nature 371, 435-438 (1994).

Burnett, T. S., & Sleeman, J. P., Uneven distribution of methylation sites within the human papillomavirus 1a genome: possible relevance to viral gene expression. Nucleic Acids Res. 12, 8847-60 (1984).

Chan, W. K., Klock, G. & Bernard, H. U. Progesterone and glucocorticoid response elements occur in the long control regions of several human papillomaviruses involved in anogenital neoplasia. J. Virol. 63, 3261-3269 (1989).

Demeret, C., Desaintes, C., Yaniv, M. & Thierry, F. Different mechanisms contribute to the E2-mediated transcriptional repression of human papillomavirus type 18 viral oncogenes. J. Virol. 71, 9343-9349 (1997).

Forrester, W. C., Fernandez, L. A., & Grosschedl, R. Nuclear matrix attachment regions antagonize methylation-dependent repression of long-range enhancer-promoter interactions. Genes Dev. 13, 3003-3014 (1999).

Francis, D. A., Schmid, S. I. & Howley, P. M. Repression of the integrated papillomavirus E6/E7 promoter is required for growth suppression of cervical cancer cells. J. Virol. 74, 2679-2686 (2000).

Futscher, B. W. et al. Role for DNA methylation in the control of cell type specific maspin expression. Nat. Genet. 31, 175-179 (2002).

Gloss, B., Bernard, H. U., Seedorf, K. & Klock, G. The upstream regulatory region of the human papillomavirus-16 contains an E2 protein independent enhancer which is specific for cervical carcinoma cells and regulated by glucocorticoid hormones. EMBO J. 6, 3735-3743 (1987).

Gloss, B., Chong, T. & Bernard, H. U. Numerous nuclear proteins bind the long control region of human papillomavirus type 16: A subset of 6 of 23 DNAsel-protected segments coincides with the location of the cell-type-specific enhancer. J.Virol. 63,1142-1152 (1989).

Harrington, M. A., Jones, P. A., Imagawa, M. & Karin, M. Cytosine methylation does not affect binding of transcription factor Sp1. Proc. Natl. Acad. Sci. USA 85, 2066-2070 (1988).

zur Hausen, H. Papillomavirus infections—a major cause of human cancers. Biochim. Biophys. Acta 1288, F55-F78 (1996).

Hendrich, B & Bird, A. Mammalian methyltransferases and methyl-CpG-binding domains: Proteins involved in DNA methylation. In DNA methylation and cancer (eds. Jones, P. A. & Vogt, P. K.) 55-74 (Springer, Berlin, 2000).

Herman, J. G. & Baylin, S. B. Promoter-region hypermethylation and gene silencing in human cancer. In DNA methylation and cancer. (eds. Jones, P. A. & Vogt, P. K.) 35-54. (Springer, Berlin, 2000).

List, H. J. et al. Methylation sensitivity of the enhancer from the human papillomavirus type 16. J. Biol. Chem. 269, 11902-11911 (1994).

May, M. et al. The E6/E7 promoter of extrachromosomal HPV16 DNA in cervical cancers escapes from cellular repression by mutation of target sequences for YY1. EMBO J. 13, 1460-1466 (1994).

Myers, G. et al. Human papillomaviruses Compendium. Los Alamos National Laboratory, Los Alamos, New Mexico, USA (1994).

'Connor, M. J., Stünkel, W., Koh, C. H., Zimmermann, H. & Bernard, H. U. The differentiation-specific factor CDP/Cut represses transcription and replication of human papillomaviruses through a conserved silencing element. J. Virol. 74, 401-410 (2000).

Okano, M., Xie, S. & Li, E. Cloning and characterization of a family of novel mammalian DNA (cytosine-5) methyltransferases. Nat. Genet. 19, 219-220 (1998).

Ozbun, M. A. & Meyers, C. Characterization of late gene transcripts expressed during vegetative replication of human papillomavirus type 31b. J. Virol. 71, 5161-5172 (1997).

Paulsen, M. & Ferguson-Smith, A. C. DNA methylation in genomic imprinting, development, and disease. J. Pathol. 195, 97-110 (2001).

Peyton C. L. et al. Determinants of genital human papillomavirus detection in a US population. J. Infect Dis. 183,1554-1564 (2001).

Remus, R. et al. Insertion of foreign DNA into an established mammalian genome can alter the methylation of cellular DNA sequences. J. Virol. 73, 1010-1022 (1999).

Rhee, I. et al. DNMT1 and DNMT3b cooperate to silence genes in human cancer cells. Nature 416, 552-556 (2002).

Robertson, K. D. & Jones, P. A. DNA methylation: past, present and future directions. Carcinogenesis 21, 461-467 (2000).

Robertson, K. D. The role of DNA methylation in modulating Epstein-Barr Virus gene expression. In: DNA methylation and cancer. (eds. Jones, P. A. &. Vogt, P. K.). 21-34. (Springer, Berlin, 2000).

Rosl, F., Arab, A., Klevenz, B. & zur Hausen, H. The effect of DNA methylation on gene regulation of human papillomaviruses. J. Gen.Virol. 74, 791-801 (1993).

Schiffman, M. H. & Brinton, L. A. The epidemiology of cervical carcinogenesis. Cancer76,1888-1901 (1995).

Schorderet, D. F. & Gartler, S. M. Analysis of CpG suppression in methylated and nonmethylated species. Proc. Natl. Acad. Sci. USA 89: 957-961(1992).

Stewart, F. J. & Raleigh, E. A. Dependence of McrBC cleavage on distance between recognition elements. Biol. Chem. 379, 611-616 (1998).

Stünkel, W., Huang, Z., Tan, S. H., O'Connor, M. & Bernard, H. U. Nuclear matrix attachment regions of human papillomavirus-16 repress or activate the E6 promoter depending on the physical state of the viral DNA. J. Virol. 74, 2489-2501 (2000).

Stünkel, W. & Bernard, H. U. The chromatin structure of the long control region of human papillomavirus type 16 represses viral oncoprotein expression. J. Virol. 73, 1918-1930 (1999).

Sugawara, K., Fujinaga, K., Yamashita, T., & Ito Y., Integration and methylation of shope papillomavirus DNA in the transplantable Vx2 and Vx7 rabbit carcinomas. Virology 131, 88-99 (1983).

Sutherland, E., Coe, L.& Raleigh, E. A. McrBC: a multisubunit GTP-dependent restriction endonuclease. J. Mol. Biol. 225, 327-348 (1992).

Tan, S. H., Leong, L. E. C., Walker, P. A. & Bernard, H. U. The human papillomavirus type 16 transcription factor E2 binds with low cooperativity to two flanking binding sites and represses the E6 promoter through displacement of Sp1 and TFIID. J. Virol. 68, 6411-6420 (1994).

Thain, A., Jenkins, O., Clarke, A. R. & Gaston, K. CpG methylation directly inhibits binding of the human papillomavirus type 16 E2 protein to specific DNA sequences. J. Virol. 70, 7233-7235 (1996).

Van Tine, B. A., Knops, J., Broker, T. R., Chow, L. T. & Moen, P. T. In situ analysis of the transcriptional activity of integrated viral DNA using tyramide-FISH. Dev. Biol. 106, 381-385 (2001).

Wettstein, F. O. & Stevens, J. G. Shope papillomavirus DNA is extensively methylated in non-virus producing neoplasms. Virology 126, 493-504 (1983).

Yoder, J. A., Walsh, C. P. & Bestor, T. H. Cytosine methylation and the ecology of intragenomic parasites. Trends Gen. 13, 335-340 (1997).

What is claimed is:

1. A method of diagnosing cervical cancer in a patient infected with human papillomavirus HPV-16 comprising the steps of:
1) providing a sample containing HPV-16 genomic DNA from the cervical cells of the patient, the HPV-16 genome having eleven CpG dinucleotides between positions 7498 and 161; and
2) determining the presence or absence of methylation of at least one of said eleven CpG sites in the HPV-16 genome in the sample, wherein the more CpG sites on which methylation is absent the more indicative it is of cervical cancer.

2. A method according to claim 1, wherein the presence of methylation at all of said eleven CpG dinucleotides is indicative of the absence of cervical cancer.

3. A method according to claim 1 wherein there are CpG dinucleotides at positions 37, 43, 52 and 58, in the HPV-16 genome, the absence of methylation in at least one of said CpG dinucleotides being indicative of the presence of at least a precursor lesion at stage CIN I.

4. A method according to claim 1, wherein the HPV-16 genome has a CpG dinucleotide at position 7862 and wherein the absence of methylation at position 7862 is indicative of at least a precursor lesion of level CIN I.

5. A method according to claim 1, wherein step 2) comprises the step of digesting the HPV-16 genome with a methylation sensitive endonuclease.

6. A method according to claim 5, wherein step 2) further comprises the step of: adding primers to the sample and effecting the PCR process, the primers being at either end of an amplicon containing at least one CpG site susceptible to cleavage by the methylation sensitive endonuclease and; analysing the results of the PCR process to determine whether said at least one CpG site was cleaved.

7. A method according to claim 6 wherein the primers are SEQ. ID NOS. 19 and 20.

8. A method according to claim 6, wherein the methylation sensitive endonuclease is McrBC endonuclease.

9. A method according to claim 1, wherein step 2) comprises selectively modifying the cytosine residue of said eleven CpG dinucleotides depending upon the methylation status of the CpG.

10. A method according to claim 9 wherein step 2) further comprises, after selectively modifying the cytosine residue, adding primers to the sample and effecting the PCR process, the primers being at either end of an amplicon containing the at least one CpG site.

11. A method according to claim 10 wherein the primers are SEQ ID NOS. 27 and 28.

12. A method according to claim 10 wherein step 2) further comprises the step of sequencing the amplicon using the primers, after the step of effecting the PCR process.

13. A method according to claim 9, wherein step 2) further comprises the step of sequencing at least a portion of the HPV-16 genome containing the at least one CpG site after the step of selectively modifying the cytosine residue of the said eleven CpG dinucleotides.

14. A method according to claim 9 wherein the step of selectively modifying the cytosine residue of said eleven CpG dinucleotides comprises adding sodium bisulfite to the sample in order to modify umnethylated cytosine residues to uracil residues.

15. A method according to claim 9, wherein step 2) further comprises the steps of: adding at least one detection probe capable of hybridising to said at least one CpG site with a first melting temperature and capable of hybridising to the site with a second, different melting temperatures when the cytosine residue of the CpG site has been modified; and monitoring the hybridisation of the detection probe to the portion of the HPV-16 genome containing the CpG site at a range of temperatures after the modifying step.

16. A method according to claim 15 further comprising the steps of: adding to the sample at least one anchor probe capable of hybridising to a position on the HPV-16 genome adjacent the at least one detection probe, the detection probe comprising one of a donor dye capable of absorbing light at a first wavelength and an acceptor dye capable of emitting light at a second wavelength, the anchor probe comprising the other of the donor dye and the acceptor dye, the donor and acceptor dyes being such that they are capable of fluorescence resonance energy transfer when they are in close proximity to one another; exposing the sample to light at the first wavelength; and detecting the emission of light at the second wavelength over the range of temperatures.

17. A method according to claim 16, further comprising the steps of: dividing the sample into eleven sub-samples; adding to each sub-sample a detection probe and a corresponding anchor probe, the detection probes being capable of hybridising to a CpG site at positions 7535, 7554, 7677, 7683, 7695, 7862, 31, 37, 43, 52 and 58, respectively, on the HPV-16 genome; exposing each sub-sample to light at the first wavelength; and detecting light emitted at the second wavelength from each sub-sample over the range of temperatures.

18. A method of assessing the progression of cervical cancer in a patient infected with HPV-16, comprising the steps of:
1) providing a sample containing the HPV-16 genomic DNA from the cervical cells of the patient; and
2) detecting the methylation state of at least the CpG dinucleotide at position 7862 of the HPV-16 genome, the lack of methylation at said dinucleotide indicating the presence of at least a precursor lesion of cervical cancer.

19. A method according to claim 18, wherein the HPV-16 genome has eleven CpG dinucleotides between positions 7498 and 161 and step 2) further comprises the steps of detecting the methylation state of said eleven CpG dinucleotides of the HPV-16 genome between positions 7498 and 161, the greater the number of dinucleotides lacking methylation indicating the more advanced progression to cervical cancer in the patient.

20. A DNA primer having a sequence consisting of a sequence selected from the group consisting of sequence ID NOS.19, 20, 27 and 28.

21. A kit for assessing the progression of cervical cancer in a patient infected with HPV-16 comprising a methylation sensitive endonuclease and a pair of primers defining either end of an amplicon within the HPV-16 genome, the amplicon containing at least one CpG site between positions 7498 and 161 of the HPV-16 genome susceptible to cleavage by the methylation sensitive endonuclease.

22. A kit according to claim 21, wherein the primers are SEQ. ID NOS: 19 and 20.

23. A kit according to claim 21 wherein the methylation sensitive endonuclease is McrBC endonuclease.

24. A kit for assessing the progression of cervical cancer in a patient infected with HPV-16 comprising:
an agent capable of selectively modifying the sequence of a CpG site depending upon the methylation status of the site; and
a pair of primers defining either end of an amplicon, the amplicon containing at least one CpG site between positions 7498 and 161 of the HPV-16 genome susceptible to modification by the agent.

25. A kit according to claim 24 wherein the pair of primers are SEQ. ID NOS. 27 and 28.

26. A kit according to claim 24, further comprising at least one detection probe, the detection probe comprising a detectable marker and being capable of hybridising to said CpG site with a first melting temperature and being capable of hybridising to the site with a second, different melting temperature when the CpG site is modified.

27. A kit according to claim 26 wherein the at least one detection probe is capable of hybridising to the CpG site at the position in the HPV-16 genome selected from the group consisting of 7535, 7554, 7677, 7683, 7695, 7862, 31, 37, 43, 52 and 58.

28. A kit according to claim 27 wherein there are eleven detection probes, the detection probes being capable of hybridising to the CpG site at positions 7535, 7554, 7677, 7683, 7695, 7862, 31, 37, 43, 52 and 58, respectively, in the HPV-16 genome.

29. A kit according to claim 26 further comprising at least one anchor probe capable of hybridising to a position on the HPV-16 genome adjacent the at least one detection probe, the anchor probe comprising a detectable marker, the detectable marker of the detection probe being selected from one of a donor dye and an acceptor dye, the detectable marker of the anchor probe being selected from the other of the donor dye and the acceptor dye, the donor and acceptor dyes being such that they are capable of fluorescence resonance energy transfer when they are in close proximity to one another.

30. A kit according to claim 29 wherein the donor dye is Fluorescein and the acceptor dye is selected from the group consisting of Light Cycler-Red 640 and Light Cycler-Red 705.

31. A kit according to claim 27 wherein the detection probe is capable of hybridising to the CpG site at position 7862 in the HPV-16 genome.

32. A kit according to claim 24 wherein the agent is selected from the group consisting of sodium bisulfite and potassium permanganate.


SEQUENCE LISTING (28 sequences)

SEQ ID NO: 1 - DNA, 22 bp, Artificial Sequence
  Primer
gtcttgttgcagatcatcaaga

SEQ ID NO: 2 - DNA, 20 bp, Artificial Sequence
  Primer
attctgaaaaactcaccccg

SEQ ID NO: 3 - DNA, 20 bp, Artificial Sequence
  Primer
gagttatacggggtgagttt

SEQ ID NO: 4 - DNA, 22 bp, Artificial Sequence
  Primer
caatggtctatgctttacatcc

SEQ ID NO: 5 - DNA, 22 bp, Artificial Sequence
  Primer
ctatggatgtaaagcatagacc

SEQ ID NO: 6 - DNA, 21 bp, Artificial Sequence
  Primer
tttccggtgtctggctctgat

SEQ ID NO: 7 - DNA, 20 bp, Artificial Sequence
  Primer
agcgaccaagatcagagcca

SEQ ID NO: 8 - DNA, 20 bp, Artificial Sequence
  Primer
ggccttgttcccaatggaat

SEQ ID NO: 9 - DNA, 20 bp, Artificial Sequence
  Primer
ggtgggttaggaattggaac

SEQ ID NO: 10 - DNA, 20 bp, Artificial Sequence
  Primer
gatgtagagggtacagatgg

SEQ ID NO: 11 - DNA, 20 bp, Artificial Sequence
  Primer
ttctacaaccccggtaccat

SEQ ID NO: 12 - DNA, 20 bp, Artificial Sequence
  Primer
gtcgccatatggttctgaca

SEQ ID NO: 13 - DNA, 20 bp, Artificial Sequence
  Primer
tggtgtcagaaccatatggc

SEQ ID NO: 14 - DNA, 24 bp, Artificial Sequence
  Primer
caacatacatacaatacttacagc

SEQ ID NO: 15 - DNA, 40 bp, Artificial Sequence
  Primer
cgtaagctgtaagtattgtatgtatgttgaattagtgttg

SEQ ID NO: 16 - DNA, 32 bp, Artificial Sequence
  Primer
ttacagctgggtttctctacgtgttcttgatg

SEQ ID NO: 17 - DNA, 40 bp, Artificial Sequence; identical to SEQ ID NO: 15
  Primer

SEQ ID NO: 18 - DNA, 28 bp, Artificial Sequence
  Primer
cctgtgggtcctgaaacattgcagttct

SEQ ID NO: 19 - DNA, 35 bp, Artificial Sequence
  Primer
tgtgtgcaaaccgttttgggttacacatttacaag

SEQ ID NO: 20 - DNA, 32 bp, Artificial Sequence; identical to SEQ ID NO: 16
  Primer

SEQ ID NO: 21 - DNA, 33 bp, Artificial Sequence
  Primer
atgcaccaaaagagaactgcaatgtttcaggac

SEQ ID NO: 22 - DNA, 32 bp, Artificial Sequence; identical to SEQ ID NO: 16
  Primer

SEQ ID NO: 23 - DNA, 38 bp, Artificial Sequence
  Primer
actacaataattcatgtataaaactaagggcgtaaccg

SEQ ID NO: 24 - DNA, 28 bp, Artificial Sequence
  Primer
cctgtgggtcctgaaacattgcagttct

SEQ ID NO: 25 - DNA, 25 bp, Artificial Sequence
  Primer
atttgatattatatttaaggttgaa

SEQ ID NO: 26 - DNA, 25 bp, Artificial Sequence
  Primer
aataattacaaaaacaaaatctaca

SEQ ID NO: 27 - DNA, 25 bp, Artificial Sequence
  Primer
tagttttatgttagtaattatggtt

SEQ ID NO: 28 - DNA, 24 bp, Artificial Sequence
  Primer
acaactctatacataactataata